(19) United States
(12) Patent Application Publication
(10) Pub. No.: US 2016/0136089 A1
(43) Pub. Date: May 19, 2016

(54) UNIT DOSE FORMULATIONS OF KETOROLAC FOR INTRANASAL ADMINISTRATION

(71) Applicant: EGALET US, INC., Wayne, PA (US)

(72) Inventors: Roger Whiting, Los Altos, CA (US); Ramachandran Thirucote, Atherton, CA (US)

(73) Assignee: EGALET US, INC., Wayne, PA (US)

(21) Appl. No.: 14/947,319

(22) Filed: Nov. 20, 2015

Related U.S. Application Data
(63) Continuation of application No. 12/483,586, filed on Jun. 12, 2009, now abandoned.
(60) Provisional application No. 61/061,522, filed on Jun. 13, 2008.
(60) Provisional application No. 61/160,254, filed on Mar. 13, 2009.

(51) Int. Cl. A61K 9/00; A61K 31/167; A61K 31/407

(52) U.S. Cl. CPC A61K 9/0043; A61K 31/407; A61K 31/167

(57) ABSTRACT

This invention relates to therapeutic compositions, particularly sprayable aqueous compositions, and unit dose formulations comprise ketorolac or a pharmaceutically acceptable salt, alone or in combination with lidocaine or a pharmaceutically acceptable salt thereof. The compositions are nasally administered to a subject in need thereof to treat pain or inflammation.

### CROSS-REFERENCE TO RELATED APPLICATION

This application claims the benefit under 35 U.S.C. §119(e) of U.S. Provisional Patent Application Ser. No. 61/061,522, filed on Jun. 13, 2008, and 61/160,254, filed on Mar. 13, 2009, both of which are incorporated herein by reference in their entirety.

### FIELD OF THE INVENTION

This invention relates to a pharmaceutical composition and a unit dose formulation of the pharmaceutical composition suitable for intranasal administration, which includes ketorolac or its pharmaceutically acceptable salts as the active analgesic and anti-inflammatory ingredient, and optionally lidocaine to reduce the sensation of stinging and to improve efficacy. This invention also relates to a therapeutic method that provides for the nasal administration of the composition to a subject to treat pain or inflammation.

### BACKGROUND OF THE INVENTION

Ketorolac or 5-benzoyl-2,3-dihydro-1H-pyrrolizine-1-carboxylic acid has the following Formula (I):

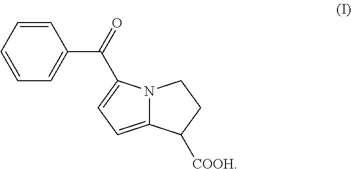 [SMILES: O=C(c1ccccc1)c1ccc2n1CCC2C(=O)O]

It has been known for several years (U.S. Pat. No. 4,089,969) and is used in human therapy as an analgesic and an anti-inflammatory as the tromethamine salt. U.S. Pat. No. 4,089,969 is incorporated herein by reference in its entirety.

Both the racemic form and each of the dextro and levo isomers of this compound are known. Many pharmaceutically acceptable salts, the most commonly used of which is the tromethamine (2-amino-2-hydroxymethyl-1,3-propanediol) salt, are also known.

Ample literature is available on ketorolac (for instance, “Ketorolac—A review of its pharmacodynamic and pharmacokinetic properties and its therapeutic potential”, Drugs 39(1): 86-109, 1990). It is described as a drug with considerably higher analgesic activity than many other non-steroidal anti-inflammatory drugs. Most significantly, it has analgesic activity comparable to that of the opiates, such as morphine, without the well-known side effects of the latter.

It is known that ketorolac can be formulated as a nasally administrable composition. See U.S. Pat. No. 6,333,044 to Recordati, which is incorporated herein by reference in its entirety. U.S. Patent Application Publication No. 2009/0042968, the content of which is incorporated hereby by reference in its entirety, describes a composition that is a combination of ketorolac and a local anesthetic for nasal administration to reduce the stinging sensation. Specifically, the composition disclosed in U.S. Patent Application Publication No. 2009/0042968 comprises an effective amount of ketorolac in combination with a pharmaceutically acceptable diluent and 4%-10% weight to volume (w/v) of a local anesthetic, e.g., lidocaine hydrochloride (preferably 5-6% w/v). Preferably the composition is a sprayable aqueous solution comprising ketorolac tromethamine present at a level of about 2.5-22.5% w/v, and lidocaine hydrochloride present at a level of 4% to 10% w/v.

### SUMMARY OF THE INVENTION

One aspect of this invention is a unit dose formulation for nasal administration to one or two nostrils comprising:

(a) greater than 12 to about 38 mg of ketorolac per nostril at a concentration of greater than 22.5% w/v; and

(b) a pharmaceutically acceptable carrier;

wherein said unit dose has a volume of 100 microliters or less per nostril.

In another aspect, the 100 microliters of composition further comprises up to about 10 mg of lidocaine at a concentration of from 4% to 10% w/v. In some embodiments, the unit dose comprises up to about 38 mg and preferably about 30 mg of ketorolac tromethamine and about 6 mg of lidocaine hydrochloride per nostril, and the volume is about 100 microliters per nostril. In some embodiments, the unit dose comprises up to about 17 mg and preferably about 15 mg of ketorolac tromethamine and about 3 mg of lidocaine hydrochloride per nostril, and the volume is about 50 microliters per nostril.

Another aspect of this invention is a composition for spraying into a human subject's nasal passage that comprises:

(a) a pharmaceutically acceptable carrier;

(b) greater than 22.5 to about 38% w/v of ketorolac, or a pharmaceutically acceptable salt (e.g., tromethamine); and

(c) optionally other pharmaceutically acceptable excipients.

Another aspect of this invention is a composition for spraying into a human subject's nasal passage that comprises:

(a) a pharmaceutically acceptable carrier;

(b) greater than 22.5 to about 38% w/v of ketorolac, or a pharmaceutically acceptable salt (e.g., tromethamine);

(c) about 4 to 10% w/v of lidocaine, or a pharmaceutically acceptable salt (e.g., hydrochloride); and

(d) optionally other pharmaceutically acceptable excipients.

Another aspect of this invention is a method for treating pain or inflammation in a subject in need of such treatment, which comprises intranasally administering the composition of this invention to the subject.

Another aspect of this invention is a method for treating pain or inflammation in a subject in need of such treatment, which method comprises administering a unit dose formulation of this invention to one nostril of the patient. In some embodiments, the method comprises administering a unit dose formulation of this invention to each nostril of the patient.

Another aspect of the invention is the composition of the invention in a vessel equipped with a device for spraying the composition into a patient's nasal passage.

Another aspect of the invention is the unit dose formulation of the invention in a single-use vessel equipped with a device for spraying the composition into a patient's nasal passage. Another aspect of the invention is the unit dose formulation of the invention in vessel equipped with a device for spraying the composition into a patient's nasal passage wherein the vessel comprises a head space with an oxygen content that is less than the normal atmospheric oxygen content.

Another aspect of the invention is the use of greater than about 22.5 to 38% w/v of ketorolac or a pharmaceutically-acceptable salt thereof, optionally in combination with about 4% to about 10% w/v of lidocaine or a pharmaceutically acceptable salt thereof, to prepare a composition for nasal administration to a subject for the treatment of pain or inflammation.

In some embodiments, the pain is the result of a trauma inflicted on the subject. In some embodiments, the pain is the result of a medical operation performed on the subject. In some embodiments, the pain is pathological. In some embodiments, the pain is neuropathic. In some embodiments, the pain is migraine or other headache pain.

These and other embodiments are described in details below.

### DETAILED DESCRIPTION OF THE INVENTION

### Definitions:

Before the compositions and methods are described, it is to be understood that the invention is not limited to the particular methodologies, protocols, and reagents described, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

In accordance with the present invention and as used herein, the following terms are defined with the following meanings, unless explicitly stated otherwise.

As used in the specification and claims, the singular form “a”, “an” and “the” include plural references unless the context clearly dictates otherwise. For example, the term “a pharmaceutically acceptable salt” includes a plurality of pharmaceutically acceptable salts, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace amount of elements of other ingredients and substantial method steps. Embodiments defined by each of these transition terms are within the scope of this invention.

The term “about” when used before a numerical designation, e.g., pH, temperature, amount, concentration, and molecular weight, including range, indicates approximations which may be varied by (+) or (−) 5%, 1% or 0.1%.

The term “greater than” when used in front of a number refers to a range that does not include the number. For example, “greater than 22.5%” does not include 22.5%.

“Ketorolac” refers to the chemical compound of 5-benzoyl-2,3-dihydro-1H-pyrrolizine-1-carboxylic acid which has the following formula (I):

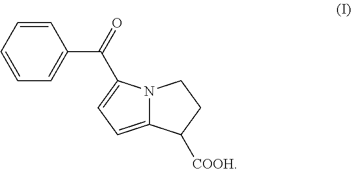 [SMILES: O=C(c1ccccc1)c1ccc2n1CCC2C(=O)O]

The name ketorolac encompasses individually or collectively the racemic mixture, a scalemic (or enantiomerically enriched) mixture, optically active compound, or a pharmaceutically acceptable salt of any of the above. Many pharmaceutically acceptable salts of ketorolac, for example ketorolac tromethamine, are known. As used herein, a racemic mixture of ketorolac is a mixture having equal amount of the two enantiomers of Formula (I). A scalemic or enantiomerically enriched mixture of ketorolac is a mixture where the amount of one of the enantiomers of Formula (I) is larger than the other enantiomer. An optically active compound may include enantiomerically enriched or enantiomerically pure compound. Enantiomerically pure compound refers to ketorolac having more than 99%, preferably 99.5%, or 99.9% of one of the enantiomers relative to the total amount of ketorolac.

“Lidocaine” refers to the chemical compound of 2-(diethylamino)-N-(2,6-dimethylphenyl)acetamide, which has the formula (II):

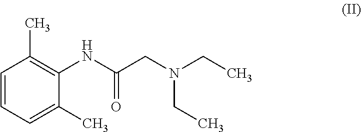 [SMILES: CCN(CC)CC(=O)Nc1c(C)cccc1C]

or a pharmaceutically acceptable salt thereof. Many pharmaceutically acceptable salts of lidocaine are known. Non-limiting examples of such salts are lidocaine hydrochloride and lidocaine methanesulphonate. As used herein, the term “lidocaine” refers to the compound or any of its pharmaceutically acceptable salts, unless otherwise indicated.

The term “subject,” “individual” or “patient” refers to a human.

The term “aggregate daily dose” refers to the total amount of drug or compound administered to a patient in a 24 hour period. The aggregate daily dose should not exceed the maximum dosing allowed by the relevant regulatory agency, such as the United States Food and Drug Administration (FDA) or the European Medicines Agency (EMEA).

The term “pharmaceutically acceptable salt” refers to pharmaceutically acceptable salts derived from a variety of organic and inorganic counter ions well known in the art and include, by way of example only, sodium, potassium, calcium, magnesium, ammonium, tromethamine, and tetraalkylammonium, and when the molecule contains a basic functionality, salts of organic or inorganic acids, such as hydrochloride, hydrobromide, tartrate, mesylate (also known as methanesulfonate), acetate, maleate, and oxalate. Suitable salts include those described in P. Heinrich Stahl, Camille G. Wermuth (Eds.), Handbook of Pharmaceutical Salts Properties, Selection, and Use; 2002.

### Unit Dose

Administering ketorolac tromethamine nasally has certain advantages over administering the compound by injection or orally. These advantages are discussed in prior art references U.S. Pat. No. 6,333,044 (“the '044 Patent”) and U.S. Patent Application Publication 2009/0042968 (“the '968 Publication”). The latter reference teaches that ketorolac tromethamine is successfully combined with a local anesthetic, e.g. lidocaine hydrochloride, to reduce the stinging effect that some patients experience with the nasal administration of ketorolac tromethamine alone. Because the '044 Patent and the '968 Publication teach the maximum concentration of ketorolac tromethamine for nasal administration to be 22.5% w/v, it was thought that higher concentrations of the compound could not be achieved. Indeed, with 4 to 6% lidocaine in the solution, it was expected that increasing the concentration of ketorolac tromethamine would increase the likelihood of precipitation and instability of the solution perhaps reducing the shelf life of the product.

Realizing that in some instances, it may be advantageous to deliver more of the active, optionally along with the lidocaine hydrochloride, in a unit dose to a patient, it was attempted to deliver more drug by increasing the volume of the solution per nostril to the patent. Although up to 200 microliters can be administered to one nostril, this large volume can lead to significant drainage of the solution from the nostril and to loss of the active ingredient, due to the limited capacity of the nostril and surface area of nasal mucosa. Thus, it was found that the amount of drug administered to a nostril may not be effectively increased simply by increasing the volume administered. It was further found that the maximum volume that can be absorbed by a nostril is about 125 microliters. Excess amount can run out of the nostril, resulting in undesirable reduced therapeutic effect and/or side effects to areas contacted by the running liquid. This can lead to reduced patient compliance. Preferably, the volume of the unit dose does not exceed about 100 microliters; with 75, 50, or 25 microliters per patient nostril providing the highest likelihood of good patient acceptance and compliance.

Surprisingly, we have now found that it is possible to prepare analgesic/anti-inflammatory intranasal formulations containing a high concentration of the active ingredient ketorolac for the treatment of pain and/or inflammation in a human subject. It is contemplated that the formulations containing high concentration of ketorolac are suitable for intranasal administration to obtain a stronger therapeutic effect than that obtained by the previously described intranasal formulations of ketorolac and yet limit the volume administered to at or below the capacity of the nostril to avoid drainage, increase the likelihood of patient acceptance and thus compliance, increase bioavailability and/or provide more reproducible pharmacokinetic profile. The high concentration also allows a high unit dosage of ketorolac to be administered to a patient in need thereof with a single spray to one or each nostril. Further, it is contemplated that the concentration of lidocaine, or a salt thereof, would not need to be increased with an increase in the concentration of ketorolac and can still effectively reduce the stinging sensation caused by the increased concentration of ketorolac.

This discovery leads to various aspects of this invention, which will be discussed hereinafter. These aspects include a novel composition particularly suited for use in a unit dosage, a novel device using the composition for nasal delivery, a method for treating pain or inflammation using the composition, and a system for delivering the composition that comprises the composition in the device with instructions for use.

In one of its aspect, the composition of this invention comprises a solution of ketorolac tromethamine that contains more than 22.5% w/v ketorolac tromethamine at a pH suitable for nasal delivery to a human subject, optionally in combination with about 4-10% w/v of lidocaine as a pharmaceutically acceptable salt.

Thus, in one embodiment, this invention relates to a unit dose formulation for nasal administration to one or two nostrils comprising

(a) greater than 12 to about 38 mg of ketorolac per nostril at a concentration of greater than 22.5% w/v; and

(b) water;

wherein said unit dose has a volume of 100 microliters or less per nostril.

In another embodiment, this invention relates to a unit dose formulation for nasal administration comprising

(a) greater than 12 to about 17 mg of ketorolac per nostril at a concentration of greater than 22.5% w/v; and

(b) water;

wherein said unit dose has a volume of about 50 microliters or less per nostril.

In some embodiments, ketorolac is ketorolac tromethamine.

In some embodiments, the volume per nostril does not exceed about 100 microliters. In some embodiments, the volume per nostril is about 50 microliters. In some embodiments, the amount of ketorolac can be increased in 1 mg increments or a part thereof. For example, in some embodiments, the unit dose formulation comprises more than 12 mg, about 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, or 38 mg, of ketorolac tromethamine per nostril.

In some embodiments, the unit dose further comprises about 2 to about 10 mg of lidocaine or a pharmaceutically acceptable salt thereof, e.g. lidocaine hydrochloride, provided that the amount of lidocaine does not exceed about 10% w/v and is more than about 4% w/v. In some embodiments, the unit dose formulation further comprises about 2, 3, 4, 5, 6, 7, 8, 9, or 10 mg of lidocaine hydrochloride.

In some embodiments, a subject is administered about 50 to 100 microliters per nostril to one or both nostrils. For example, for an acute episode of migraine attack, two single sprays of about 100 microliters, each containing about 30 mg of ketorolac can be administered to each nostril to provide for a unit dose of about 60 mg of ketorolac, which is expected to provide fast relief of the pain, and/or to stop the pain from aggravating and to prevent or to eliminate other symptoms associated with migraine, such as nausea and sensitivity to light and sound. In some embodiments, a unit dose of about 30 mg can be administered by two sprays of about 50 microliters containing about 15 mg of ketorolac, one to each nostril so that all drug can be retained in the nostril(s). This reduced volume is expected to provide increased bioavailability and/or better pharmacokinetics yet maintain the therapeutic effect.

It is contemplated that such a high dosage of ketorolac would deliver higher analgesic or anti-inflammatory efficacy yet would not increase the side effects significantly. It is further contemplated that notwithstanding the higher dose of ketorolac in the formulations of this invention, the amount of lidocaine present in the formulation will inhibit stinging during application while also minimizing or eliminating numbing in the nasal mucosa and/or the throat.

In some embodiments, the unit dose is in the form of an aqueous solution.

In some embodiments, the unit dose comprises ketorolac tromethamine. In some embodiments, the unit dose comprises about 30 mg of ketorolac tromethamine and has a volume of about 100 microliters per nostril. In some embodiments, the unit dose comprises about 15 mg of ketorolac tromethamine and has a volume of about 50 microliters per nostril. In some embodiments, the unit dose is for administration to two nostrils which unit dose comprises about 15 mg of ketorolac tromethamine and has a volume of about 50 microliters per nostril. In some embodiments, the unit dose is for administration to one nostril, which unit dose comprises about 15 mg of ketorolac tromethamine and has a volume of about 50 microliters. In some embodiments, the unit dose is for administration to two nostrils which unit dose comprises about 30 mg of ketorolac tromethamine and has a volume of about 100 microliters per nostril. In some embodiments, the unit dose is for administration to one nostril which unit dose comprises about 30 mg of ketorolac tromethamine and has a volume of about 100 microliters.

In some embodiments, the unit dose further comprises lidocaine hydrochloride. In some embodiments, the unit dose comprises about 6 mg of lidocaine hydrochloride per nostril. In some embodiments, the unit dose comprises about 3 mg of lidocaine hydrochloride per nostril.

The unit dose for any particular patient will depend upon a variety of factors known in the art, including the type and severity of the pain or inflammation to be treated, the age, body weight, general health, sex and diet, renal and hepatic function of the patient, and the time of administration, and will generally be in accordance with the advice of the attending physician. The unit dose as described above can be contained in a single container designed to hold a volume of the pharmaceutical composition such that single or multiple administrations can be administered from that container.

In some embodiments, the unit dose formulation further comprises a chelator, i.e. a substance that binds primarily di- or tri valent metallic ions (e.g. calcium) that might interfere with the stability or activity of the active ingredient. Chelators are known to those of skill in the art by referring to the recent edition of “Remington's Pharmaceutical Sciences.” A preferred chelator is sodium ethylenediamine tetraacetic acid (sodium EDTA), USP. In some embodiments, the unit dose comprises about 0.001 to about 1 mg of disodium edetate. In some embodiments, it comprises about 0.01 to about 0.1 mg of disodium edetate. In some embodiments, it comprises about 0.02 mg of disodium edetate.

In some embodiments, the pH of the unit dose is about 4.5 to about 8, or about 4.8 to about 7.5. In some embodiments, the pH of the unit dose is about 7.2. In some embodiments, the pH is adjusted by a pharmaceutically acceptable base, such as sodium hydroxide.

In some embodiments, the pH of the unit dose is further adjusted and/or maintained by a sufficient amount of a pH buffering agent, such as potassium phosphate monobasic (KH₂PO₄), potassium phosphate dibasic (K₂HPO₄) or potassium phosphate (K₃PO₄), optionally in combination with sodium hydroxide. In some embodiments, the unit dose comprises about 0.68 mg of potassium phosphate monobasic.

In some embodiments, the unit dose formulation comprises about 30 mg of ketorolac tromethamine, about 0.02 mg of disodium edetate, about 0.68 mg of potassium phosphate monobasic, sodium hydroxide to adjust pH to 7.2 and water to about 100 microliters per nostril.

In some embodiments, the unit dose formulation comprises about 30 mg of ketorolac tromethamine, about 6 mg of lidocaine hydrochloride, about 0.02 mg of disodium edetate, about 0.68 mg of potassium phosphate monobasic, sodium hydroxide to adjust pH to 7.2 and water to about 100 microliters per nostril.

In some embodiments, the unit dose formulation comprises about 15 mg of ketorolac tromethamine, about 0.01 mg of disodium edetate, about 0.34 mg of potassium phosphate monobasic, sodium hydroxide to adjust pH to 7.2 and water to about 50 microliters per nostril.

In some embodiments, the unit dose formulation comprises about 15 mg of ketorolac tromethamine, about 3 mg of lidocaine hydrochloride, about 0.01 mg of disodium edetate, about 0.34 mg of potassium phosphate monobasic, sodium hydroxide to adjust pH to 7.2 and water to about 50 microliters per nostril.

In some embodiments, the unit dose formulation is contained in a vessel equipped with a device for spraying the composition into the nasal passage of a subject, wherein the composition is an aqueous solution. In some embodiments, the vessel further comprises a metering chamber. In some embodiments, the metering chamber is coupled with the spraying device. An example of a device that can be used for spraying the composition into a nasal passage of the subject is disclosed in U.S. application Ser. No. 12/404,250, filed on Mar. 13, 2009, entitled, “Device for intranasal Administration,” and is incorporated herein by reference in its entirety. In some embodiments, the vessel comprises a head space with a reduced oxygen content. In some embodiments, the head space comprises equal to or less than 10%, 8%, or 5% v/v oxygen.

In some embodiments, the metering chamber holds about 50 microliters for a single spray. In another embodiment, the metering chamber holds about 100 microliters for a single spray. In some embodiments, the volume measured by the metering chamber is adjustable.

In some embodiments, the unit dose formulation is in combination with a label instruction providing for administration of about 15 to about 30 mg of ketorolac per nostril. In some embodiments, the unit dose formulation is in combination with a label instruction providing for administration of about 25 to about 60 mg of ketorolac per dose. In some embodiments, the unit dose formulation is in combination with a label instruction providing for administration of about 30 or about 60 mg of ketorolac per dose.

### Composition

In another aspect of this invention, provided are intranasal formulations suitable for providing the desired high unit dose of ketorolac to achieve better analgesic/anti-inflammatory therapeutic effects without exceeding the absorption capacity of nostril. The formulations of this invention are designed to employ a high concentration of the active ingredient ketorolac.

In some embodiments, the intranasal formulations of the invention comprise concentrations of ketorolac, or a pharmaceutically acceptable salt, ranging from greater than 22.5 to about 38% w/v, for example about 24%, 25%, 26%, 27%, 28%, 29%, 30%, 31%, 32%, 33%, 34%, 35%, 36%, 37%, or 38% w/v, based on the final formulation. In some embodiments, ketorolac is ketorolac tromethamine. In some embodiments, the composition further comprises lidocaine, or a pharmaceutically acceptable salt, ranging from about 4% to 10%, for example about 4%, 5%, 6%, 7%, 8%, 9%, or 10% w/v. In some embodiments, lidocaine is lidocaine hydrochloride.

The composition of this invention allows a patient to administer a large amount of ketorolac with just one spray comprising a maximum of 100 microliters of the composition to one or each of the nostrils. In a preferred embodiment, the composition of this invention contains 30% w/v of ketorolac and provides to a patient 60 mg of ketorolac when 100 microliters of solution containing 30 mg of ketorolac is given to each nostril of the patient. It is contemplated that a higher amount of ketorolac administered to a patient would produce more effective analgesic and/or anti-inflammatory relief and/or be useful in treating more severe pains without significant leakage. This may be beneficial for treating a migraine pain and/or preventing the deterioration of an acute migraine attack. In another preferred embodiment, the composition comprising 30% w/v of ketorolac provides to a patient 30 mg of ketorolac when 50 microliters of solution containing 15 mg of ketorolac is given to each nostril of the patient. The smaller volume will further minimize leakage, provide improved bioavailability and/or pharmacokinetics.

It has been found that ketorolac produces several degradation products, such as the 1-keto analog having Formula (III) and the chemical name of 5-benzoyl-2,3-dihydro-1H-pyrrolizin-1-one, in solution.

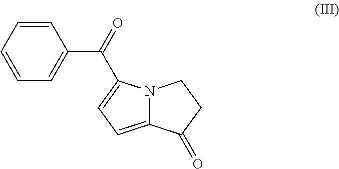 [SMILES: O=C1CCn2c1ccc2C(=O)c1ccccc1]

The amount of 1-keto analog produced is generally proportional to the amount of ketorolac in solution and available oxygen (e.g., in the head space of the container, such as those described in U.S. patent application Ser. No. 12/404,250, filed on Mar. 13, 2009, entitled, “Device for Intranasal Administration,”). It has been found that a higher concentration of ketorolac can result in a concentration of the 1-keto analog that is outside an acceptable level, which may cause safety concerns. It has further been found that the formulations of this invention may be stored under refrigeration conditions or under air with reduced oxygen content (e.g., no more than 10% v/v) to allow a high dose of ketorolac to be administered intranasally to a patient without compromising the safety requirements.

In some embodiments, the compositions of this invention comprise a combination of ketorolac and lidocaine. As described in details in US Patent Application Publication No. 2009/0042968, the addition of lidocaine to the 15% w/v ketorolac composition was found to provide several unexpected advantageous synergistic effects. First, the combination substantially reduces the stinging sensation caused by ketorolac. Second, while lidocaine is a local anesthetic that is known to cause numbness, such numbness is substantially absent or reduced when lidocaine is combined with ketorolac. Third, combination of 5 to 6% w/v of lidocaine with ketorolac have been found to decrease the time for ketorolac to reach its Cmax in a subject's plasma (the “Tmax”), providing a subject with faster and better pain relief. Surprisingly, when the concentration of ketorolac is increased to up to 38%, the amount of lidocaine can be maintained at a level of 4-10% or preferably 5-6% and yet still inhibits the stinging sensation of ketorolac. Although the concentration of ketorolac has increased relative to the lidocaine, it is contemplated that the benefits of decreased Tmax will still be achieved by the combination of lidocaine with ketorolac.

Lidocaine and its pharmaceutically acceptable salts, such as lidocaine hydrochloride, have poor solubility in water under the physiological pH ranges of 4.5 to 7.2 although it is soluble to greater than 6% w/v at the lower pH ranges of 2.5 to 4.5. Certain other local anesthetics, such as benzocaine, also exhibit low solubility at physiological pH although soluble at low pH. For example, benzocaine hydrochloride is soluble in water and the solution has a pH of about 1.5. In the presence of 15% ketorolac, however, precipitation was observed for both a 6% and a 10% benzocaine solution. Such precipitation was not re-dispersed by heating or adjusting the pH. Surprisingly, lidocaine is found to be soluble in 15% or 30% of ketorolac tromethamine solution with a pH of 7.2. Without being limited to any theory, it appears that the ketorolac synergistically assists in the solubilization of lidocaine tromethamine and that such synergy continues even when the amount of ketorolac is increased. This makes lidocaine uniquely suitable for reducing the stinging sensation associated with intranasal administration of ketorolac.

In some embodiments, the invention provides a composition which is an aqueous solution suitable for nasal administration to a subject, which solution comprises
(a) greater than 22.5% w/v to about 38% w/v of ketorolac,
(b) water, and
(c) a pharmaceutically acceptable pH adjuster to maintain the solution at a pH of about 4.5 to 8.

In some embodiments, the invention provides a composition which is an aqueous solution suitable for nasal administration to a subject, which solution comprises:
(a) greater than 22.5% w/v to about 38% w/v of ketorolac or a pharmaceutically acceptable salt thereof,
(b) about 4% w/v to about 10% w/v of lidocaine or a pharmaceutically acceptable salt thereof,
(c) water, and
(d) a pharmaceutically acceptable pH adjuster to maintain the solution at a pH of about 4.5 to 8.

In some embodiments, ketorolac is as a racemic mixture.

In some embodiments, the composition comprises about 25 to 35% w/v of ketorolac or a pharmaceutically acceptable salt thereof. In some embodiments, the composition comprises about 28 to 32% w/v of ketorolac or a pharmaceutically acceptable salt thereof. In some embodiments, the composition comprises about 30% w/v of ketorolac or a pharmaceutically acceptable salt thereof. In some embodiments, the composition comprises ketorolac tromethamine. In some embodiments, the composition comprises about 30% w/v of ketorolac tromethamine.

In some embodiments, the composition comprises lidocaine hydrochloride. In some embodiments, the composition comprises about 4% w/v to about 7.5% w/v of lidocaine hydrochloride. In some embodiments, the composition comprises about 5-6% w/v lidocaine hydrochloride. In some embodiments, the composition comprises about 6% w/v lidocaine hydrochloride.

In some embodiments, the composition further comprises a chelator. In some embodiments, the chelator is disodium edetate.

In some embodiments, the pH of the composition is about 4.5 to 8. In some embodiments, the pH is about 4.8 to 7.5. In some embodiments, the pH is about 7.2.

In some embodiments, the pH is adjusted by a pharmaceutically acceptable base. In some embodiments, the pharmaceutically acceptable base is sodium hydroxide.

In some embodiments, the composition further comprises a pH buffer to create optimum pH conditions for both product stability and tolerance (pH range about 4 to about 8; preferably about 6.0 to 7.5). Suitable buffers include without limitation tris (tromethamine) buffer, phosphate buffer, etc. Preferably potassium phosphate NF, potassium phosphate monobasic, or potassium phosphate bibasic, or a combination thereof is used to maintain the pH to 7.2. In some embodiments, the composition comprises up to about 2% w/v of potassium phosphate monobasic. In some embodiments, the composition comprises about 0.68% w/v of potassium phosphate monobasic.

In some embodiments, the composition is contained in a vessel suitable for spraying the solution into a subject's nostril. In some embodiments, the vessel having a size suitable to contain about 0.1 to 4 mL of the solution. In some embodiments, the vessel having a size suitable to contain about 0.2 to 2.4 mL of the solution.

In some embodiments, the composition is in combination with a label instruction providing for administration of 50 to about 100 microliters of the solution per nostril. In some embodiments, the label instruction provides for administration of about 100 microliters of the solution per nostril. In some embodiments, the label instruction provides for administration of about 50 microliters of the solution per nostril.

In some embodiments, the composition comprises:

(a) about 30% w/v of racemic ketorolac tromethamine,

(b) about 0.01% w/v to about 0.1% w/v of disodium edetate,

(c) up to about 2% w/v of potassium phosphate monobasic,

(d) sodium hydroxide to adjust the to 7.2, and

(e) water to 100% w/v.

In some embodiments, the composition comprises:

(a) about 30% w/v of racemic ketorolac tromethamine,

(b) about 5% w/v to about 6% w/v lidocaine hydrochloride,

(c) about 0.01% w/v to about 0.1% w/v of disodium edetate,

(d) up to about 2% w/v of potassium phosphate monobasic,

(e) sodium hydroxide to adjust the pH to 7.2, and

(f) water to 100% w/v.

In some embodiments, the composition comprises about 5% w/v of lidocaine hydrochloride. In some embodiments, the composition comprises about 6% w/v of lidocaine hydrochloride.

Another aspect of the invention is the composition of the invention in a vessel equipped with a device for spraying the composition into a patient's nasal passage. In some embodiments, the vessel is further equipped with a metering chamber to measure a desired amount of the composition to be sprayed to the patient's nasal passage. In some embodiments, the metering chamber is coupled with the spraying device so that a patient can simultaneously measure and spray a desired amount (e.g. a unit dose) of the composition. In some embodiments, the metering chamber is able to measure from about 50 to about 125 microliters of liquid. In some embodiments, the metering chamber is able to measure from about 50 to about 100 microliters of liquid. In some embodiments, the metering chamber is adjustable. In some embodiments, the metering chamber is able to measure about 50 microliters of liquid. In some embodiments, the metering chamber is able to measure about 100 microliters of liquid.

The unit dose of the compositions of this invention is usually administered one, two, three, or four times a day, providing an amount generally efficacious in treating moderate to severe pain, whether of a pathological or neuropathic origin, such as trauma-inflicted pain, post-operative pain, migraine, and the like. In general, a subject that is 18 to 65 years old could receive up to the maximum allowed daily dose. For example, the dose may be an intranasal administration of ketorolac per day of 100 microliters or less per nostril of a 30% w/v ketorolac solution. In some embodiments, the composition can be given up to 4 times per day.

It is contemplated that in cases of acute migraine attacks, the high unit dosages enabled by the compositions of this invention would allow a relief of the pain and/or inhibit the progression of the pain and other symptoms of migraine so that, preferably, the patient would not experience significant migraine pains or related symptoms after the composition is administered once or twice, and avoid the need of repeated administration of ketorolac during one migraine episode.

A subject that is an adolescent or is older than 65 could receive less ketorolac, for example, by intranasal administration of 50 microliters per nostril of a 30% w/v or by intranasal administration of 100 microliters of a 30% w/v ketorolac formulation to only one nostril or 50 microliters of a 30% w/v ketorolac formulation to each nostril. Children 12 and under would receive appropriately less. It is contemplated that the nasal absorption capacity of a child is smaller than that of an adult, thus a lesser volume, e.g., 50 microliters, to one or each nostril should be administered to children to avoid discharge.

It is understood that the exact amount is dependent upon the attending physician's advice, and will be based the age or weight of the patient, severity of the pain and other factors known in the art.

The pharmaceutical compositions and unit dose formulations of this invention may optionally comprise one or more pharmaceutically acceptable excipients, including a pharmaceutically acceptable carrier (or diluent). Of course, the selection of the particular excipients depends on the desired formulation dosage form, i.e., on whether a solution to be used in drops or as a spray is desired or whether a suspension, ointment or gel to be applied directly to the nasal cavity is desired. In any case, the invention enables the preparation of single-dose or multi-dose dosage forms, which ensure application of an optimum quantity of drug.

The preferred carrier for the formulations according to the invention is water, preferably sterile water, and other excipients may be added if desired.

In addition to aqueous, oil or gel diluents, other diluents which may be used in the compositions according to the invention comprise solvent systems containing ethyl alcohol, isopropyl alcohol, propylene glycol, polyethylene glycol, mixtures thereof or mixtures of one or more of the foregoing with water.

Other excipients include chemical enhancers such as absorption promoters. These include fatty acids, bile acid salts and other surfactants, fusidic acid, lysophosphatides, cyclic peptide antibiotics, preservatives, carboxylic acids (ascorbic acid, amino acids), glycyrrhetinic acid, o-acylcarnitine. Preferred promoters are diisopropyladipate, POE(9) lauryl alcohol, sodium glycocholate and lysophosphatidyl-choline which proved to be particularly active.

If present, excipients such as oil, gel, chemical enhancers, including absorption promoters, etc. should be in an amount that does not adversely affect the homogeneity and sprayability of the solution.

The compositions of the invention can also contain a compatible preservative that ensures the microbiological stability of the active ingredient. Suitable preservatives include without limitation, methyl paraoxybenzoate (methyl paraben), propyl paraoxybenzoate (propyl paraben), sodium benzoate, benzyl alcohol, and chlorobutanol. In some embodiments, the ketorolac intranasal compositions do not contain a preservative.

The bacterial load in the compositions of this invention preferably does not exceed 100 colony forming units (CFUs) and more preferably does not exceed about 50 CFUs.

Illustrative formulations may contain the following ingredients and amounts (w/v) in addition to ketorolac, lidocaine and water.

| Ingredient | Broad Range (%) | Preferred Range (%) |
|---|---|---|
| Chelator (1) | 0.001-1 | 0.01-0.1 |
| Preservative (2) | 0-2 | 0-0.25 |
| Absorption promoter (3) | 0-10 | 0-10 |
| Gelling polymer (4) | 0-5 | 0-3 |
| Co-solvent (5) | 0-99 | 0 |

(1) E.g., sodium EDTA
(2) E.g., methyl paraoxybenzoate or propyl paraoxybenzoate or mixtures thereof
(3) E.g., sodium glycocholate
(4) E.g., sodium carboxymethyl cellulose (sodium CMC)
(5) E.g., glycerol

It will be appreciated by those of ordinary skill that ingredients such as sodium CMC and polymers designated as CARBOPOL exist in many types differing in viscosity. Their amounts are to be adjusted accordingly. Different adjustments to each formulation may also he necessary including omission of some optional ingredients and addition of others. It is thus not possible to give an all-encompassing amount range for each ingredient, but the optimization of each preparation according to the invention is within the skill of the art. The presence of the excipients should be in an amount that does not adversely affect the homogeneity and sprayability of the solution.

### Methods

Compositions of the invention are administered to a patient in need thereof by contacting the patient's nasal passage, with a unit dose or an amount of the composition sufficient to result in absorption of ketorolac by the patient to reduce the pain and/or inflammation experienced by the patient. This is preferably carried out by spraying a solution, as described herein, into the nasal passage(s) of the patient from a vessel that is equipped with a device (e.g., an atomizer) for producing a spray (e.g. atomized particles). The device produces a mist or suspension of fine liquid particles that are inhaled by the patient into her or his nasal passage(s) from which it is rapidly absorbed into the bloodstream to effect its analgesic and anti-inflammatory action. The volume administered should not exceed the maximum absorption capacity of the nostril to avoid drug loss through drainage. Appropriate vessels and spray devices are available to one of skill in the art by referring to “Remington's Pharmaceutical Sciences.” One source for such vessels is Ing. Erich Pfeiffer GmbH, Radolfzell, Germany. Another source is Valois, 50 avenue de l'Europe, 78164 MARLY-LE-ROI, France.

In another aspect, this invention provides methods for treating pain or inflammation in a subject in need of such treatment, comprising intranasally administering a pharmaceutical composition or a unit dose formulation of this invention as described above to one or each nostril of a patient.

In some embodiments, the unit dose formulation is administered once, twice, three times or four times a day.

In some embodiments, the method is for treating pain.

In some embodiments, the pain is the result of a trauma inflicted on the subject. In some embodiments, the pain is the result of a medical operation performed on the subject.

In some embodiments, the pain is pathological. In some embodiments, the pain is neuropathic. In some embodiments, the pain is migraine or other headache pain.

The following example of a formulation for the intranasal administration of ketorolac serves to illustrate the invention without limiting its scope.

### EXAMPLE 1

This example provides a description for making compositions for nasal administration in accordance with the invention. A solution was prepared in accordance with the proportions shown in Table 1.

TABLE 1

| Ingredients | Concentration (% w/v) |
|---|---|
| Ketorolac tromethamine, USP | 30 |
| Lidocaine hydrochloride | 6 |
| Sodium edetate, NF | 0.02 |
| Potassium phosphate monobasic, NF | 0.68 |
| Sodium hydroxide (2M) | (q.s. to pH 7.2) |
| Water for Injection USP (q.s.) | to 100 |

### APPENDIX OF PRODUCT NAMES AND EXAMPLES OF COMMERCIAL SOURCES

KETOROLAC TROMETHAMINE: Union Quimico Farmaceutico, S.A., Spain
HYDROXYPROPYLCELLULOSE (KLUCEL®) Dow Chemical Co, Midland Mich. USA
HYDROXYPROPYLMETHYLCELLULOSE (METHOCEL®) Dow Chem. Co, Midland Mich.
HYDROXYETHYLCELLULOSE (NATROSOL®) Hercules Inc, Wilmington Del. USA
SODIUM CARBOXYMETHYLCELLULOSE (BLANOSE®) Hercules Inc, Wilmington Del.
CARBOPOL®: BF Goodrich Chemical Co., Cleveland, Ohio, USA
POLYCARBOPHIL: BF Goodrich Chemical Co., Cleveland, Ohio, USA
ETHYL ALCOHOL: Eastman Chemical Products Inc., Kingsport, Tenn., USA
ISOPROPYL ALCOHOL: Baker Chemical Co., New York, N.Y., USA
PROPYLENE GLYCOL: Dow Chemical Co., MIDLAND, Mich., USA
POLYETHYLENE GLYCOL: BASF Wyndotte Corp., Parsippany, N.J., USA
DIISOPROPYLADIPATE: Croda, Goole, North Humerside, UK
SODIUM GLYCOCHOLATE: Sigma Chemical Company, St. Louis, Mo., USA
LYSOPHOSPHATIDYLCHOLINE: American Lecithin, Long Island, N.Y., USA
METHYLPARAOXYBENZOATE (NIPAGIN): BDH Chemical Ltd, Poole, Dorset, UK
PROPYLPARAOXYBENZOATE: BDH Chemical Ltd, Poole, Dorset, UK
SODIUM BENZOATE: Pfizer Inc., New York, N.Y., USA.
BENZYL ALCOHOL: BDH Chemical Ltd, Poole Dorset, UK
BENZALCONIUM CHLORIDE: ION Pharmaceuticals, Covina, Calif., USA
SODIUM EDTA: Grace And Co., London, UK.
POE(9)LAURYL ALCOHOL: BASF Wyndotte Corp, Parsippany, N.J., USA
GLYCEROL: Dow Chemical Co., Midland, Mich., USA
SODIUM CHLORIDE: Aldrich Chemie, Stanheim, Germany
GLUCOSE: Roquette Ltd, Tunbridge Wells, Kent, UK

1.-61. (canceled)

62. A drug delivery device for ketorolac comprising a vessel containing a unit dose of a ketorolac formulation for nasal administration to one or two nostrils of a subject, wherein the formulation comprises an aqueous composition of ketorolac at a concentration of from greater than 22.5% w/v to 38 5% w/v, and wherein said unit dose has a volume of from 50 to 100 microliters per nostril and contains from about 12 mg to about 38 mg of ketorolac per nostril.

63. The drug delivery device of claim 62, wherein the unit dose has a volume 100 microliters per nostril.

64. The drug delivery device of claim 63, wherein the unit dose contains 30 mg of ketorolac per nostril.

65. The drug delivery device of claim 62, wherein the unit dose has a volume of 50 microliters per nostril.

66. The drug delivery device of claim 65, wherein the unit dose contains 15 mg of ketorolac per nostril.

67. The drug delivery device of claim 62, wherein the formulation further comprises lidocaine at a concentration of about 10% w/v or less and the unit dose contains from about 4 mg to about 10 mg of lidocaine per nostril.

68. The drug delivery device of claim 67, wherein the unit dose contains from about 5 mg to about 6 mg of lidocaine per nostril.

69. The drug delivery device of claim 62, wherein the formulation further comprises a chelator.

70. The drug delivery device of claim 62, wherein the formulation has a pH of about 4.5 to 8.

71. The drug delivery device of claim 62, wherein the vessel comprises a headspace with a reduced oxygen content.

72. The drug delivery device of claim 62, wherein the vessel comprises a headspace with an oxygen content equal to or less than 10% w/v.

73. The drug delivery device of claim 62, wherein the vessel is equipped with a device for spraying a unit dose of the formulation into one or two nostrils of the subject.

74. The drug delivery device of claim 73, wherein the vessel further comprises a metering chamber able to measure from about 50 to about 100 microliters of liquid.

75. The drug delivery device of claim 62, provided with instruction for administering from about 15 to about 30 mg of ketorolac per nostril.

76. The drug delivery device of claim 62, provided with instruction for administering from about 30 to about 60 mg of ketorolac per unit dose.

77. The drug delivery device of claim 62, wherein the formulation comprises about 30 mg of ketorolac tromethamine, about 6 mg of lidocaine hydrochloride, about 0.02 mg of disodium edetate, about 0.68 mg of potassium phosphate monobasic, sodium hydroxide to adjust pH to about 7.2, and water to make 100 microliters, wherein the unit dose is about 100 microliters per nostril.

78. The drug delivery device of claim 62, wherein the formulation comprises about 15 mg of ketorolac tromethamine, about 3 mg of lidocaine hydrochloride, about 0.01 mg of disodium edetate, about 0.34 mg of potassium phosphate monobasic, sodium hydroxide to adjust pH to about 7.2, and water to about 50 microliters, wherein the unit dose is about 50 microliters per nostril.

79. A method for treating pain or inflammation in a subject in need thereof, comprising intranasally administering a unit dose of a ketorolac formulation from a vessel according to claim 62, from 1 to 4 times a day.

80. The method of claim 79, wherein the unit dose is administered to one nostril of the subject.

81. The method of claim 79, wherein the unit dose is administered to two nostrils of the subject.
